(19) United States
(12) Patent Application Publication
(10) Pub. No.: US 2026/0225081 A1
(43) Pub. Date: Aug. 6, 2026

(54) PROCESS FOR CONTINUOUS HYDROGENATION OF AQUEOUS LEVULINIC ACID TO GAMMA-VALEROLACTONE AND ITS TANDEM CONVERSION TO HIGH OCTANE GASOLINE RANGE HYDROCARBONS

(71) Applicant: ECO-OIL MILJÖBRÄNSLEN I SVERIGE AB, Skellefteå (SE)

(72) Inventors: Jyri-Pekka MIKKOLA, UMEÅ (SE); Ajaikumar SAMIKANNU, UMEÅ (SE); Lakhya Jyoti KONWAR, UMEÅ (SE); William SILJEBO, BJURHOLM (SE); Kent VAN KLINT, SKELLEFTEÅ (SE)

(21) Appl. No.: 19/152,382

(22) PCT Filed: Jan. 29, 2024
(86) PCT No.: PCT/EP2024/052108

§ 371 Date: Jul. 30, 2025

(30) Foreign Application Priority Data
Feb. 1, 2023 (SE) 2350094-5

(51) Int. Cl. B01J 23/46; B01J 8/00; B01J 8/02; B01J 21/18; B01J 27/195; B01J 35/30; B01J 35/61; B01J 35/63; C07C 1/207; C07D 307/33; C10G 3/00

(52) U.S. Cl. CPC B01J 23/462; B01J 8/001; B01J 8/0278; B01J 8/0285; B01J 21/18; B01J 27/195; B01J 35/393; B01J 35/615; B01J 35/633; C07C 1/2078; C07D 307/33; C10G 3/44; C10G 3/54; B01J 2208/00539; B01J 2208/00548; C07C 2521/12

(57) ABSTRACT

A Ru-loaded carbon material with characteristic surface chemistry (Bronsted acidic phosphate groups, Lewis basic N-groups selected from the group consisting of Lewis basic pyridinic, pyrrolic and quaternary N-groups and oxygen functional groups) is shown to exhibit excellent activity, selectivity and stability upon hydrogenation of aqueous levulinic acid (0.47 M and 0.95 M) to γ-valerolactone with near stoichiometric H₂ under mild conditions (80-95° C.). The material demonstrated remarkable stability in a fixed-bed reactor under continuous flow conditions, maintaining high activity, and γ-valerolactone selectivity during ~1250 h time on stream operating at 3-5 bar, 80-95° C. and low H₂/levulinic acid ratio (4-17). The exceptional catalytic performance and stability of the multifunctional Ru-catalyst was attributed to the unique surface chemistry of carbon support that stabilized the Ru nanoparticles on the surface nitrogen and oxygen defects, and promoted 4-hydroxypentanoic acid cyclization over acidic phosphate and RuOx/RuO₂ sites.

### FIELD OF THE INVENTION

The invention relates to a method for hydrogenation of levulinic acid to γ-valerolactone with H₂. It further relates to a catalyst for use in the method. γ-valerolactone can if desired further be converted to gasoline range hydrocarbons.

### BACKGROUND

Levulinic acid is a well-known product of hexose sugar hydrolysis/dehydration and it is inexpensively obtained by the decomposition of cellulosic feedstock's. Levulinic acid is an attractive platform material for production of for instance γ-valerolactone. Among the various levulinic acid derivatives, γ-valerolactone, in particular, is commercially important owing to its usefulness as a solvent, food additive, fuel component, and as a precursor for high value chemicals (linear α-olefins, esters) and fuel range synthetic hydrocarbons.

Traditionally, γ-valerolactone has been obtained by the tandem hydrogenation-dehydration of levulinic acid under pressurized H₂ (1-3 MPa). The catalytic hydroconversion of levulinic acid or its derivatives have been demonstrated with both homogeneous and heterogeneous noble and non-noble metal catalysts. Ru (Ruthenium) appears to be the most active, owing to its unique ability to selectively hydrogenate the ketonic C═O groups. Even so, elevated temperatures (≥150° C.) or acid-co catalysts are needed to promote conversion of intermediate hydroxyl acid (4-hydroxypentanoic acid) and afford selective transformation of levulinic acid γ-valerolactone. Consequently, the commercial usefulness of Ru based catalysts has been plaged due to deactivation issues resulting from (a) the leaching of Ru species through the formation of Ru-carboxylate complexes with levulinic acid (pKa ~4.59) in liquid phase, and (b) sintering and coking of Ru-nanoparticles in vapour phase systems. Another drawback of processes operating at elevated H₂ pressure and reaction temperature is the reduced γ-valerolactone selectivity due to the formation of overhydrogenation by-products such as 2-methyltetrahydrofuran and pentane-1,4-diol. Similarly, at low temperatures levulinic acid hydrogenation produces an intermediate hydroxyl acid (4-hydroxypentanoic acid) as the main product; the dehydration step is kinetically less favourable at low temperatures relative to hydrogenation step. As such elevated temperature or acid as co-catalysts are needed to afford high γ-valerolactone selectivity/yield in liquid phase hydroconvresion of levulinic acid (or its derivatives) [RSC Adv., 2017, 7, 44082, ACS Catal. 2014, 4, 4, 1171-1181]. In fact, in view of the benefits of low temperature operations (low-energy consumption and minimization of sintering and coking), researchers have employed both mineral acids and solid acids (Amberlyst®-15, Amberlyst-70, niobium phosphate, and niobic acid etc.) as co-catalysts for energy efficient hydrogenation of levulinic acid to γ-valerolactone [Green Chem., 2012,14, 688-694, ChemSusChem. 10 (14) (2017) 2891-2896].

Hence, the development of practical, efficient and stable bifunctional Ru catalysts by incorporating non-reducible and stable acids sites for selective low temperature hydrogenation of aqueous levulinic acid to γ-valerolactone has until now remained a major technical challenge.

U.S. Pat. No. 7,741,527 discloses a method for dimerization of olefin, comprising use of a solid phosphoric acid catalyst, comprising phosphoric acid, a compound that forms phosphoric acid by hydrolysis, or a mixture of both, supported on a carrier.

U.S. Pat. No. 8,148,553 discloses a method involving conversion of levulinic acid to γ-valerolactone by contacting the levulinic acid with a heterogeneous catalyst comprising Ru/C.

CN 102658131 discloses a ruthenium-based catalyst for preparing γ-valerolactone from levulinic acid, including an active component and a catalyst carrier. The active component is ruthenium 1-10 wt %. A co-catalyst as a group VIII element or a group I element with maximum 10 wt % may be present. The catalyst support can be a carbon material (for instance activated carbon, carbon black, carbon nanotubes or carbon nanofibers), an oxide (for instance alumina, silica, titania or their binary mixed oxide) or a molecular sieve (for instance MCM, ZSM, SBA or faujasite). The catalyst can be prepared using a solvent-free microwave-assisted pyrolysis method. The catalyzed reaction can be carried out with high-pressure hydrogen (1-5 MPa) at a temperature in the range 70-120° C. during 1-10 hours.

CN 108745401 discloses a nitrogen and phosphorus doped porous carbon-rhodium phosphide catalyst as well as a preparation method and application thereof. Polyhydric alcohol, a rhodium source and substances containing a nitrogen and phosphorus compound are dispersed into water; stirring is performed to form mixed sol; one-step high-temperature calcination is used for preparation to obtain a composite nanometer material with the rhodium phosphide loaded on porous carbon, wherein a porous carbon carrier is modified by nitrogen and phosphorus in the compound; rhodium phosphide nanoparticles are loaded on the nitrogen and phosphorus modified porous carbon, wherein the rhodium phosphide nanoparticles have the size 4 to 8 nm.

CN 114433163 discloses an in-situ modified and pore-adjustable biochar-loaded ruthenium catalyst, a preparation method of the in-situ modified and pore-adjustable biochar-loaded ruthenium catalyst and application of the in-situ modified and pore-adjustable biochar-loaded ruthenium catalyst in lignin.

### SUMMARY

One object of the present invention is to obviate at least some of the disadvantages in the prior art and provide an improved catalyst for hydrogenation of aqueous levulinic acid streams to γ-valerolactone.

In a first aspect there is provided a catalytic materials based on a material comprising carbon, the catalytic material is loaded with Ru, wherein the surface of the catalytic material comprises a) Brønsted acidic phosphate groups, b) Lewis basic N-groups selected from the group consisting of Lewis basic pyridinic, pyrrolic and quaternary N-groups, and c) oxygen functional groups.

In a second aspect there is provided a system comprising a catalyst bed comprising the catalytic material as described above, a heater and thermocouple, a pump for feeding levulinic acid to the catalytic bed, a pressure regulator for regulating the pressure in the catalytic bed/system, a valve for regulating inflow of H₂.

In a third aspect there is provided a method for converting levulinic acid to γ-valerolactone comprising use of the catalytic material according to any one of claims 1-7 or the system according to any one of claims 8-10, the method comprising the steps of contacting the catalyst with an aqueous solution of levulinic acid and hydrogen gas (H₂).

In fourth aspect there is provided γ-valerolactone or γ-valerolactone/4-hydroxypentanoic acid mixtures manufactured using the catalytic material or the system or the method as described above.

In a fifth aspect there is provided a C₈₊ olefin manufactured with the method as described above.

In a sixth aspect there is provided a method for preparing a catalytic material as described above, the method comprising the steps of contacting an organic polymer comprising amine groups with phosphoric acid followed by a subsequent step of carbonizing the material under elevated temperature under self-generated atmosphere to provide a carbon based material wherein a surface of the material comprises a) Brønsted acidic phosphate groups, b) Lewis basic N-groups selected from the group consisting of Lewis basic pyridinic, pyrrolic and quaternary N-groups, and c) oxygen functional groups, where after a final loading step is performed where the material is mixed with an aqueous solution of a ruthenium salt where after the ruthenium salt is reduced so that small particles comprising elemental ruthenium are loaded on the carbon based material.

Further embodiments of the present invention are defined in the appended dependent claims, which are explicitly incorporated herein.

One advantage is that the stability is extended. The long term stability is resulting from the enhanced stabilization of Ru-nanoparticles over a highly nitrogen doped carbon surface.

The conversion and selectivity is maintained and comparable or better than Ru catalysts according to the state of the art.

There is the possibility of tuning levulinic acid conversion and γ-valerolactone yield/selectivity by simple adjustment of flow rate of liquid feed (liquid/weight hourly space velocity), gaseous H₂ (H₂/levulinic acid mole ratio) and reaction temperature and pressure.

Another advantage is the possibility to work with aqueous levulinic acid with concentrations ranging from about 0.5-1.0 M, representing realistic levulinic acid feeds obtained from biomass processing.

The leaching of Ru is from the catalyst material is very limited.

The temperature and operating pressure can be kept relatively low giving low-energy consumption and minimization of sintering and coking of the catalyst.

### BRIEF DESCRIPTION OF THE DRAWINGS

Aspects and embodiments will be described with reference to the following drawings in which:

FIG. 1 shows an experimental set-up for continuous hydrogenation of aqueous levulinic acid to γ-valerolactone

FIG. 2 shows a FE-SEM image and the corresponding EDX micro-element mapping (C, O, N, P and Ru) of Ru/CNP500 catalyst

FIG. 3 shows the effect of Ru loading (1-5 wt %) on the NH₃-TPD profiles of Ru/CNP500 catalyst (IW denotes Ru/CNP500 catalyst obtained by incipient wetness impregnation and H₂ treatment at 400° C.)

FIG. 4 shows (a) levulinic acid conversion vs. normalized time (time*mmol/g_{Ru}) and (b) conversion vs. γ-valerolactone selectivity as function of Ru loading for Ru/CNP500. Reaction conditions: 0.25 g levulinic acid, 5 ml deionised H₂O, 15 mg catalyst (Ru/CNP500, Ru loading 1-5 wt %), 5.5±1 bar initial H₂, 400 rpm (stirring rate)

FIG. 5 shows the influence of (a) catalyst acidity (phosphate) upon catalytic performance (γ-valerolactone selectivity) at room temperature and (b) reaction temperature upon γ-valerolactone selectivity. Reaction conditions: 0.25 g levulinic acid, 5 ml deionised H₂O, 15 mg catalyst, 5.5±1 bar initial H₂, 400 rpm (stirring rate).

FIG. 6 shows the catalyst stability (γ-valerolactone yield) as a function of (a) reuse (batch experiment) and, (b) time on stream (fixed-bed experiment) for the multifunctional Ru/CNP500 catalyst, in comparison to physical mixtures of (Ru/Csigma+Amberlyst 15) and (Ru/Csigma+NbOPO₄/Amberlyst 15). Batch reaction conditions: 0.25 g levulinic acid, 5 ml deionised H₂O, 15 mg catalyst (@0.34 mol % Ru, 5 wt % Ru loading), 5.5±1 bar initial H₂, 80° C., 4 h, 400 rpm (stirring rate); Fixed-bed reaction conditions: 80° C., 5 bar, 0.48 M aqueous levulinic acid, H₂/levulinic acid (molar) ratio ≥10, WHSV 0.11-0.12 h-1 (with respect to 0.48 M aqueous levulinic acid).

FIG. 7 shows the catalytic performance of Ru/CNP500 catalyst (5 wt % Ru) upon continuous hydrogenation of aqueous levulinic acid during 52 days' time on stream under varying reaction conditions.

FIG. 8 shows TEM images and corresponding particle size distribution of 5 wt. % Ru/CNP500 (top-fresh and bottom-after 52 days' time on stream)

FIG. 9 shows (a) NH₃-TPD pattern of 5 wt % Ru/CNP500 after 52 days' time on stream, and (b) N₂-adosrption-desoprtion isotherms and the corresponding pore size distribution plots (inset) of Ru/CNP500 before and after 52 days' continuous operation.

FIG. 10 shows high resolution (a) C1s and Ru3d, (b) Ols, (c) N1s and (d) P2p XPS spectra of fresh Ru/CNP500 and spent Ru/CNP500 after 52 days' time on stream (5% wt Ru).

FIG. 11 shows a simplified experimental set-up for tandem conversion of levulinic acid derived aqueous γ-valerolactone into fuel range hydrocarbons. R0 (hydrogenation reactor, 80-95° C., 3.5-5 bar), R1 (decarboxylation reactor, 380° C., 1 bar) and R2 (oligomerization reactor, 170-200° C., 1 bar), MFC (Mass flow controller), S1, S2 and S3 (gas liquid separator) FIG. 12 shows tandem conversion of 9 wt % aqueous γ-valerolactone/4-hydroxypentanoic acid mixture (85-96 mol % γ-valerolactone and 4-15 mol % 4-hydroxypentanoic acid) produced over multifunctional Ru/CNP500 catalyst into fuel range hydrocarbons (C8+ branched olefins) at atmospheric pressure with a single pass dual-bed reactor system comprising commercial Silica-alumina (1st bed) and commercial Solid phosphoric acid (2nd bed) as decarboxylation and oligomerization catalyst, respectively. Reaction conditions: 380° C. (1st bed, 10 g pellets made with 30% alumina), 170-200° C. (2nd bed, 20 g pellets), 1 bar, WHSV 0.12 h-1 with respect to γ-valerolactone and 4-hydroxypentanoic acid fed into the 1st bed.

### DETAILED DESCRIPTION

Before the invention is disclosed and described in detail, it is to be understood that this invention is not limited to particular configurations, process steps and materials disclosed herein as such configurations, process steps and materials may vary somewhat. It is also to be understood that the terminology employed herein is used for the purpose of describing particular embodiments only and is not intended to be limiting since the scope of the present invention is limited only by the appended claims and equivalents thereof.

It must be noted that, as used in this specification and the appended claims, the singular forms “a”, “an” and “the” include plural referents unless the context clearly dictates otherwise.

The following terms are used throughout the description and the claims.

All percentages and ratios are calculated by weight unless otherwise stated.

“Amount” of particles or other material on a surface is herein often given as μg/cm². This is a suitable way of expressing the amount since the applied layer is very thin. For calculating the amount, the area of the object is measured and the amount per area is calculated.

“Bransted acid” is a molecule (or ion) capable of donating a proton.

“Lewis base” is an atomic or molecular species where the highest ° Occupied molecular orbital (HOMO) is highly localized. Examples include but are not limited to conventional amines such as ammonia and alkyl amines as well as pyridine and its derivatives. Further examples include but are not limited to amines of the formula NH₃₋ₓRₓ where R=alkyl or aryl, pyridine and its derivatives, phosphines of the formula PR₃₋ₓAₓ, where R=alkyl, A=aryl, compounds of O, S, Se and Te in oxidation state −2, including water, ethers and ketones.

“Nanoparticle” is a particle of matter with a diameter in the range 1-100 nm. For irregular particles the largest particle size is taken as the diameter. For a sphere the largest size is the diameter.

Oxygen functional groups are chemical functional groups comprising an oxygen atom and include for instance alcohols, ethers, aldehydes, ketones, and carboxylic acids as well as a variety of derivatives of the carboxylic acids such as the amides, esters, and acid halides.

There is in one embodiment disclosed a highly porous multifunctional Ru/C catalyst incorporating unique surface properties (oxygen functional groups, strongly Brønsted acidic, non-reducible phosphate sites and abundant Lewis basic N-sites) as a catalyst for selective low temperature tandem hydrogenation-dehydration of aqueous levulinic acid to γ-valerolactone. There is disclosed a chitin (bio-waste) ( ) derived low-cost, multifunctional Ru/C catalyst, from extended stability tests conducted in a continuous flow fixed-bed reactor (~1250 h time on stream) and using a low H₂:levulinic acid mole ratio (≤17) under mild conditions (3-3.5 bar and 80-95° C.).

In the first aspect there is provided a catalytic material based on a material comprising carbon, the catalytic material is loaded with Ru, wherein the surface of the catalytic material comprises a) Brønsted acidic phosphate groups, b) Lewis basic N-groups selected from the group consisting of Lewis basic pyridinic, pyrrolic and quaternary N-groups, and c) oxygen functional groups.

In one embodiment, the content of Ru is 0.1-10 wt %. In one embodiment, the content of Ru is 1-6 wt %. In one embodiment, the Ru is present in the form of nanoparticles comprising Ru, preferably with average particle size d_{p,H2} in the interval 1.5-5 nm as measured by H₂ pulse chemisorption. In another embodiment the Ru is present in the form of nanoparticles with average particle size d_{p,H2} in the interval 1.5-3.75 nm nm as measured by H₂ pulse chemisorption. In yet another embodiment the Ru is present in the form of nanoparticles with average particle size d_{p,H2} in the interval 1.7-3.7 nm as measured by H₂ pulse chemisorption.

In one embodiment, the NH₃-acidity is more than 1.5 mmol/g, measured by NH₃-TPD (temperature programmed desorption) (total 70-450° C.).

In one embodiment, the CO₂-basicity is more than 0.03 μmol/g, measured by CO₂-TPD (temperature programmed desorption) (total 30-150° C.).

In one embodiment, the specific surface area is at least 300 m²/g. The specific surface area is measured by gas adsorption by the BET method according to ISA 9277:2010.

In one embodiment, the pore volume is at least 0.3 cm³/g.

In one embodiment, the catalytic material is based on a carbonized organic material. In one embodiment, the catalytic material is based on carbonized chitin.

In one embodiment, the Brønsted acidic phosphate groups comprises NbOPO₄.

In one embodiment the catalytic material comprises Brønsted acidic phosphate (—OPO₄ sites) groups covalently bound to the carbon. In one embodiment the catalytic material comprises 0.18 mmol/g Brønsted acidic phosphate (—OPO₄ sites) groups covalently bound to the carbon.

It is intended that the Brønsted acidic phosphate groups and the oxygen functional groups are different. Thus the oxygen functional groups under c) do not include Brønsted acidic phosphate groups under a). It is intended that the oxygen functional groups in c) are not Brønsted acidic phosphate groups. The Brønsted acidic phosphate groups under a) do not fall under the oxygen functional groups in c). The oxygen functional groups in c) do not include Brønsted acidic phosphate groups.

In the second aspect there is provided a system comprising a catalyst bed comprising the catalytic material as described above, a heater and thermocouple, a pump for feeding levulinic acid to the catalytic bed, a pressure regulator for regulating the pressure in the catalytic bed/system, a valve for regulating inflow of H₂.

In one embodiment, the system comprises a valve for regulating inflow of N₂.

In one embodiment, the system comprises a gas-liquid separator for separating obtained liquid product from unreacted H₂.

In the third aspect there is provided a method for converting levulinic acid to γ-valerolactone comprising use of the catalytic material as described above or the system as described above, the method comprising the steps of contacting the catalyst with an aqueous solution of levulinic acid and hydrogen gas (H₂).

In one embodiment, the H₂/levulinic acid mole ratio is in the interval 3-20, preferably 4-17.

In one embodiment, the pressure during reaction is in the interval 1.5-10 bar, preferably 2-7 bar.

In one embodiment, the temperature is above 70° C. but below the boiling point of the aqueous levulinic acid solution at the pressure at which the reaction is carried out, preferably in the interval 80-95° C.

In one embodiment, the catalyst is contacted with the aqueous solution of levulinic acid and hydrogen gas batch-wise in a reaction vessel. In one embodiment, the catalyst is contacted with the aqueous solution of levulinic acid and hydrogen gas batch-wise in a high pressure reaction vessel.

In one embodiment, the catalyst is contacted in a fixed-bed reactor under continuous flow conditions of aqueous solution of levulinic acid and hydrogen gas.

In one embodiment, the levulinic acid is made from a raw material comprising cellulose.

In one embodiment, the γ-valerolactone or γ-valerolactone/4-hydroxypentanoic acid mixture is further converted into C₈₊ olefins in a cascade process at atmospheric pressure with a single pass, dual-bed reactor system comprising commercial silica-alumina (1^{st} bed) and solid phosphoric acid (2^{nd} bed).

In the fourth aspect there is provided γ-valerolactone manufactured using the catalytic material as described in any of the aspects and embodiments above.

In the fifth aspect there is provided a C₈₊ olefin manufactured as described above.

In a sixth aspect there is provided a method for preparing a catalytic material as described above, the method comprising the steps of contacting an organic polymer comprising amine groups with phosphoric acid followed by a subsequent step of carbonizing the material under elevated temperature under self-generated atmosphere to provide a carbon based material wherein a surface of the material comprises a) Brønsted acidic phosphate groups, b) Lewis basic N-groups selected from the group consisting of Lewis basic pyridinic, pyrrolic and quaternary N-groups, and c) oxygen functional groups, where after a final loading step is performed where the material is mixed with an aqueous solution of a ruthenium salt where after the ruthenium salt is reduced so that small particles comprising elemental ruthenium are loaded on the carbon based material.

In one embodiment, the organic polymer comprising amine groups is chitin.

In one embodiment, the material is carbonized under elevated temperature of at least 500° C. in an atmosphere comprising an elevated concentration of N₂ compared to the normal atmosphere. Normal atmosphere is normal air with a content of about 78 wt %.

Other features of the invention and their associated advantages will be evident to a person skilled in the art upon reading the description and the examples. It is understood that the disclosed embodiments can be freely combined with all other embodiments as long as it is not clearly contradictory.

It is to be understood that this invention is not limited to the particular embodiments shown here. The following examples are provided for illustrative purposes and are not intended to limit the scope of the invention since the scope of the present invention is limited only by the appended claims and equivalents thereof.

### EXAMPLES

### Chemicals

NaOH (99%, Sigma-Aldrich), H₃PO₄ (85%, VWR), HCl (37%, Merck), chitin flakes (from shrimp shells, Sigma-Aldrich), ammonium dihydrogen phosphate (99.9%, Merck), Amberlyst® 15 (dry, H⁺ form, Sigma-Aldrich) Ru/C (5%, Sigma-Aldrich), levulinic acid (97%, Sigma-Aldrich), γ-valerolactone (99%, Sigma-Aldrich), niobium(V)chloride (99.8%, ACROS), ruthenium chloride (RuCl₃.×H₂O, abcr GmbH, 36% Ru) were obtained from commercial sources and used without further purification.

### Catalytic Materials

The bifunctional carbon support containing nitrogen and phosphate sites were obtained by the well-known one step phosphoric acid activation described in literature. This method is well known and widely practiced for obtaining high surface are nitrogen and phosphate self doped carbon from nitrogen rich bio-wastes including chitin, for instance in [JP Mikkola, V Kent, W Siljebo, LJ Konwar, A Samikannu, Hydrothermal method for producing renewable paraffinichydrocarbons, 2021, EP3841186A1; L. J. Konwar, J. P Mikkola, Carbon support effects on metal (Pd, Pt and Ru) catalyzed hydrothermal decarboxylation/deoxygenation of triglycerides Appl. Catal. A. General, 638, 2022, 118611 & A Samikannu, LJ Konwar, P Maki-Arvela, JP Mikkola, Renewable N-doped active carbons as efficient catalysts for direct synthesis of cyclic carbonates from epoxides and CO₂ Appl. Catal. B, 241,2019, 41-51]. In a typical synthesis procedure, 10 g of chitin flakes were soaked with 50 wt % aqueous phosphoric acid solution in 1:2 (w/w) ratio and aged for 24 h under ambient conditions. In the next step, the chitin-phosphoric acid mixture was transferred into an alumina crucible, loaded into a muffle furnace (Nabertherm, LT15) and activated at 500° C. (heating rate 3° C./min) for 1 h in the self-generated atmosphere. The obtained materials were ground to powder and thoroughly washed with hot deionized water (90° C.) until neutral pH and dried in an oven at 100° C. for 24 h to produce the bifunctional carbon supports (CNP500) containing nitrogen (basic), oxygen and phosphorus (acidic) functional groups.

The basic carbon material (CN650) containing only nitrogen sites without phosphorus doping was obtained by the direct carbonization of chitin flakes at 650° C. according to the following procedure. In a typical synthesis procedure, 10 g gram chitin flakes were loaded onto an alumina boat, transferred into a tubular furnace (Carbolite) and carbonized under 50 ml/min N₂ flow at 350° C. (heating rate 2.5° C./min) for 2 h, the obtained carbon materials were further carbonized at 650° C. for 1.5 h (heating rate 5° C./min, 50 ml/min N₂). The resulting carbon materials were powdered and washed with deionized water and dried overnight at 100° C. to obtain the basic carbon support functionalized with nitrogen functional groups (CN650).

The carbon support containing oxygen functional groups and trace amount of phosphates (CPcomm) was obtained from by phosphoric acid washing of powdered activated carbon obtained from Fisher scientific. The Ru loaded catalysts (Ru/CN650, Ru/CNP500, and Ru/CPcomm) were prepared by employing the well-known deposition-precipitation method described in literature, for instance in Pham, T. N., Samikannu, A., Rautio, AR. et al. Catalytic Hydrogenation of D-Xylose Over Ru Decorated Carbon Foam Catalyst in a SpinChem® Rotating Bed Reactor. Top Catal 59, 1165-1177 (2016). This deposition-precipitation method is well known and widely practiced. The synthesis procedure for supported catalyst was as follows: 1 g support was added to 50 ml deionised water under stirring, at room temperature. After stirring for 1 h, an appropriate amount of ruthenium chloride (aqueous) solution (2.5 mg/ml corresponding to metal loading of 1-5 wt %) was added to the mixture/suspension and stirred for 2 h, at room temperature. Next, 0.1 M NaOH was slowly added to the reaction mixture to adjust the pH of the solution to ~9.5. After further stirring the resulting mixture for 1 h, a calculated amount of freshly prepared NaBH₄ solution (nNaBH₄/nRu=15/1) was rapidly added into this solution and further stirred for 1 h at room temperature. To obtain the desired catalytic materials, contents of the reaction mixture were filtered and extensively washed with deionised water until free from Cl⁻ ions and dried under a stream of N₂ at 70° C. for 24 h. A reference catalyst Ru/CNP500_IW was also prepared following the incipient wetness impregnation and H₂ reduction technique.

The Nb based support, NbP-bulk (NbOPO₄) was prepared according to the procedures described in literature (hydrolysis of niobium(V)chloride in 12 M HCl in the presence of stoichiometric amount of ammonium dihydrogen phosphate, followed by calcination at 400° C.) according to the method described in [Samikannu A., Konwar L. J., Rajendran K., Lee C. C., Shchukarev A., Virtanen P., Mikkola J-P. Highly dispersed NbOPO4/SBA-15 as a versatile acid catalyst upon production of renewable jet-fuel from bio-based furanics via hydroxyalkylation-alkylation (HAA) and hydrodeoxygenation (HDO) reactions, Appl. Catal. B, 272, 2020, 118987]. The Ru loaded catalyst Ru/NbP-bulk catalyst was obtained by incipient wetness impregnation of RuCl₃ according to the procedures described elsewhere. Prior to use the catalytic material was reduced at 400° C., under a stream of H₂.

The actual metal Ru loadings measured by ICP-OES, which were identical to the designed loadings in all the catalysts. The commercial ruthenium catalyst (Ru/CSigma) with 5 wt % ruthenium loading and oxygen functionalized active carbon supports were obtained from Sigma Aldrich and used as received.

### Catalytic Reaction Procedures

Batch experiments were performed in a custom made mini autoclave (~13 mL) assembled from Swagelok parts with stoichiometric amount of H₂. The reactor was equipped with a pressure gauge, inlet and outlet valves. In a typical experiment, after loading the reactor with desired amount of catalyst (15-30 mg), levulinic acid (0.25 g) and water (5 g), the reactor was sealed and purged up to 5 times with pure H₂ gas. In the next step, the reactor was pressurized to the desired initial H₂ pressure (5.5±1 bar or ~2.5 mmol H₂). To initiate the reaction, the contents of the reactor was heated to the desired reaction temperature (85° C.) in a sand bath under magnetic stirring (500 rpm) for an appropriate duration. After completed reaction, the reactor was cooled under tap water, the residual H₂ carefully released and the liquid contents centrifuged to separate the catalyst.

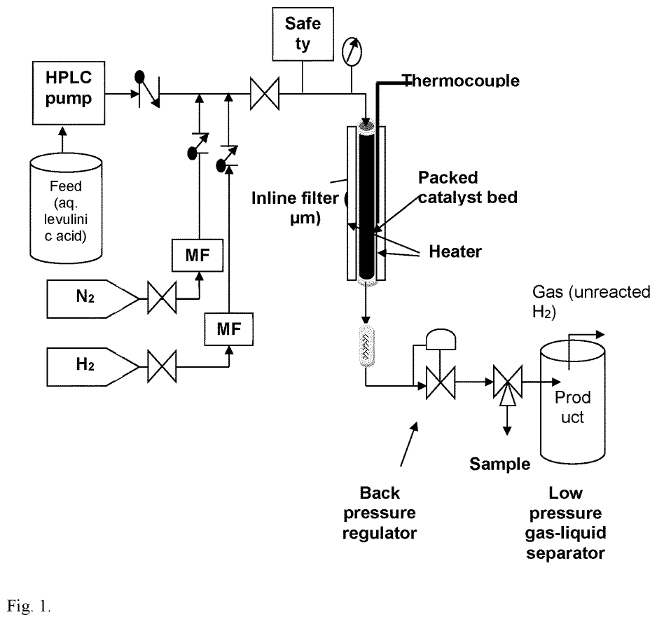

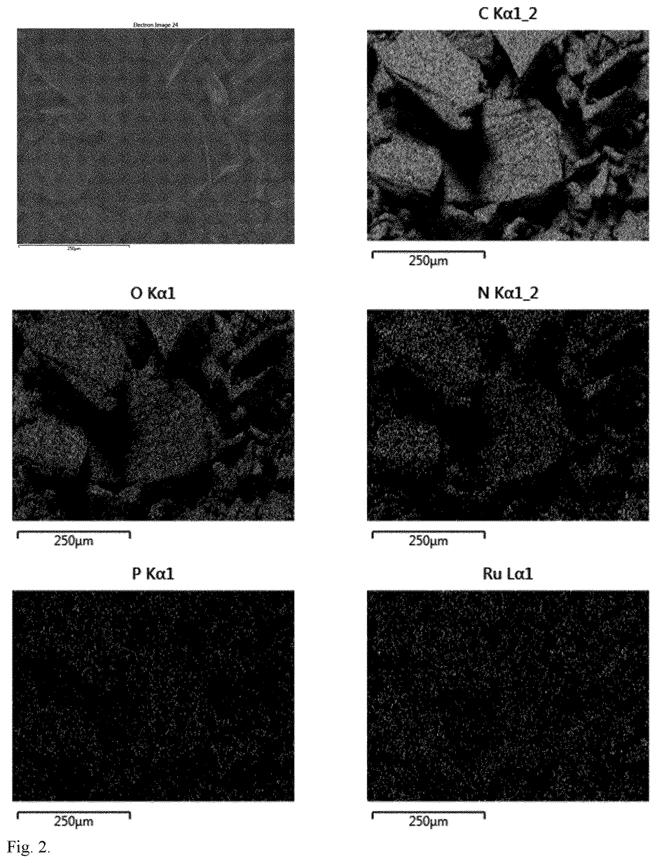

In a typical continuous experiment, the dry catalyst powder (1-1.6 g) was packed into the custom made tubular down flow mini fixed bed-reactor (12 mm OD, 10 mm ID and length 16 cm) assembled from high pressure 316 stainless-steel tubing, tube fittings and valves purchased from Swagelok (FIG. 1). The reactor temperature was maintained (80-95° C.) using aluminium blocks heated by band heaters and the total pressure (3-5 bar) controlled using a Back Pressure Regulator (Equilibar U3L Series Precision Back Pressure Regulator). The liquid feed (0.47 or 0.95 M levulinic acid) was pumped continuously into the reactor using an HPLC pump (Perkin Elmer series 200 micro pump), while H₂ was simultaneously supplied using a mass flow controller (Bronkhorst EL-FLOW® Select). In a typical experiment, the system was initially degassed with N₂ at 10 mL min⁻¹ flow for 1 h, then the flow was switched to H₂ and the flow rate adjusted for obtaining the desired H₂: levulinic acid (molar ratio) and reactor heated to the desired temperature, after which the liquid feed was pumped continuously.

For determining the conversion and selectivity, the liquid aliquots/solutions collected from batch and continuous flow experiments were periodically analyzed by HPLC (Agilent Technologies 1200 Series, RID detector, Aminex HPX-87H Column) and GC-FID (Agilent 7820A GC, HP-5MS capillary column, 30 m length, 0.25 mm internal diameter, 0.25 μm film thickness), the quantification of individual components in the liquid-phase mixture were based on calibration curves obtained with commercially available compounds. To check for Ru leaching, liquid aliquots were also periodically analyzed by ICP-OES on a PerkinElmer (OPTIMA 2000 DV) instrument.

### Catalytic Materials Characterization

Different samples of Ru-catalyst were prepared using porous supports of varying surface acidities. Table 1 presents a summary of the textural and surface acid-base properties of the different support materials used in the work. From the data presented in Table 1, it is seen that NbP-bulk presented the most acidic surface with abundant strong Brønsted sites (Nb₂O₃ and NbOPO₄), while the carbon materials (CPcomm, CNP500, and CN650) presented a highly functionalized surface comprising of neutral, weakly acidic, moderately Brønsted acidic, and Lewis basic sites.

The carbon support obtained by chitin pyrolysis (CN650) presented surface comprising of natural, weakly acidic and basic sites (attributed to the presence of —COOH, —OH, pyridinic nitrogen, pyrrolic/pyridonic nitrogen and N oxide of pyridinic nitrogen species), while the carbon material obtained by phosphoric acid activation of chitin (CNP500) presented a multifunctional surface chemistry comprising of neutral-to-weakly acidic oxygen functional groups, strongly Brønsted acidic phosphate sites and basic pyridinic, pyrrolic/pyridonic, quaternary and N oxide of pyridinic nitrogen sites (Table 1). In contrast, the commercial active carbon (CPcomm) presented an acidic surface comprising of only oxygen and trace amount of phosphorus functional groups.

Table 2, summarizes the textural properties, dispersion, and average metal particle size of supported Ru-catalysts using the different supports discussed above, in comparison to the commercial Ru/CSigma. The Ru/NbP-bulk catalyst was obtained by incipient wetness impregnation of RuCl₃ according to the following procedure. In a typical synthesis, a calculated amount of ruthenium chloride precursor solutions (the volume of the precursor solution was calculated to be equal to the pore volume of NbP-bulk) was added to the support (NbP-bulk) under vigorous agitation. The impregnated sample was dried overnight at 100° C., followed by further reduction at 400° C. for 3 h under 10 ml/min of H₂ flow (ramp 1° C./min). In contrast, the carbon based catalytic materials were synthesized following the standard deposition-precipitation of RuCl₃ followed by reduction with NaBH₄.

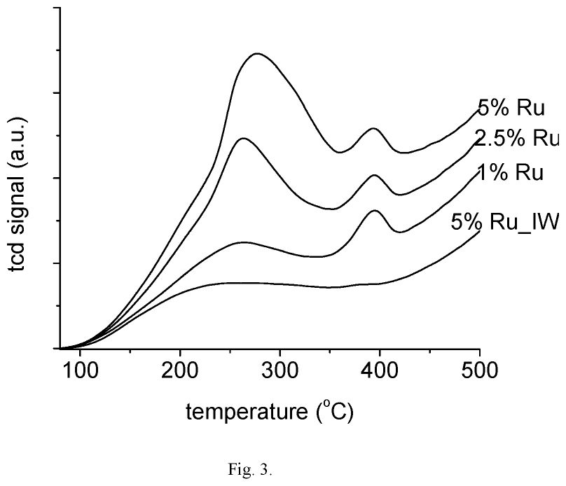

From the data presented in Table 2, the importance of the carbon support surface chemistry on metal particle dispersion and average metal particle size is clearly seen. The presence of surface functional groups, especially Brønsted acidic (Nb₂O₅, and NbOPO₄) and Lewis basic nitrogen sites was clearly favourable for the formation of uniformly dispersed and small metal (Ru) nanoparticles. The effect was clearly noticeable for NbP-bulk, CN650 and CNP500 whereupon highly dispersed (37-76%) and ultra small Ru nanoparticles (1.76-3.63 nm) were obtained at 2.5-5 wt % Ru loading (Table 2). The presence of well dispersed Ru species and the uniform distribution of surface functional groups comprising of nitrogen, oxygen and phosphate sites could be confirmed from the EDX microelement (Ru, P, O, C and N) mapping images (FIG. 2). Furthermore, from the NH₃-acidity values presented in Table 2 and NH₃-TPD profiles in FIG. 3, a positive effect of Ru nanoparticles immobilization upon catalyst surface acidity is clearly observed. The enhanced acidity of Ru-loaded catalysts could be attributed to the presence of Lewis acidic of RuO₂/RuOₓ species.

### Catalytic Activity in Batch

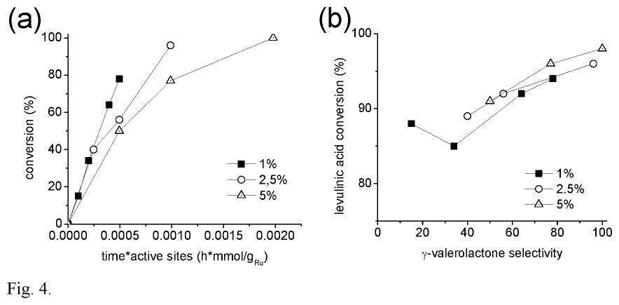

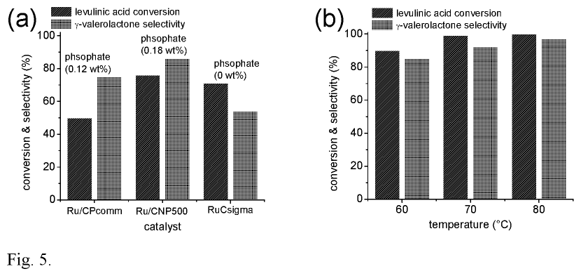

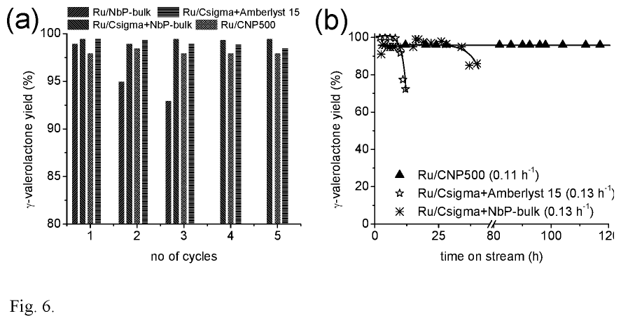

In order to evaluate the effect of catalyst properties upon hydrogenation of aqueous levulinic acid, preliminary catalytic tests were conducted in batch mode using near stoichiometric amount of H₂ (5.5±1 bar or ca. 2.5 mmol) at 80° C. The results from batch experiments presented in Table 3, confirm the positive effect of catalyst acidity in terms of γ-valerolactone selectivity. The activity/selectivity trends observed with the multifunctional Ru-catalysts incorporating phosphate sites (Ru/NbP-bulk, Ru/CNP500 and Ru/CPcomm) were comparable with the selectivity trends of Brønsted acid (Amberlyst 15 and NbOPO₄) promoted Ru/Csigma catalyzed hydrogenation of levulinic acid, in agreement with the role of strong/moderately Brønsted acidic surface acid sites as a co-catalysts for the dehydration of 4-hydroxypentanoic acid (Scheme 2). Interestingly, we observed a positive correlation between levulinic acid conversion, and γ-valerolactone selectivity with increasing Ru loading (FIG. 4), in agreement with the acidity trends presented in Table 2 and FIG. 3, and the Lewis acidity of RuO₂/RuOₓspecies. In addition, the method of catalyst preparation also had impact on the acidity of supported Ru catalysts, which in turn influenced the catalyst activity (conversion, γ-valerolactone yield and selectivity). In this work, the catalysts prepared by incipient wetness impregnation and H₂ reduction (Ru/CNP500_IW) presented ca. two-fold lower activity than the Ru/CNP500 catalyst obtained by deposition-precipitation technique resulting from 3.5 fold lower NH₃ acidity of the former (FIG. 3, Table 2 and 3). The Lewis acidity of RuO₂/RuOₓ species also accounts for the rather high (90%) γ-valerolactone selectivity observed at 80° C. for the Ru/Csigma catalyst incorporating only oxygen functional groups, while the presence of surface Lewis basic sites were clearly detrimental for γ-valerolactone production as observed for Ru/CN650 (Entry 2, Table 3). The influence of catalyst surface acidity on γ-valerolactone selectivity was more clearly noticeable at room temperature, whereupon Ru/Csigma presented ca. ~1.6 fold lower γ-valerolactone selectivity than the multifunctional Ru/CNP500 and Ru/CPcomm catalysts incorporating phosphate sites (FIG. 5(a) and Table 3). The positive effect of reaction temperature on γ-valerolactone selectivity could also be observed from plots in FIG. 5(b). It is also important to highlight here that in all catalytic experiments only stoichiometric amount of H₂ was consumed producing γ-valerolactone and 4-hydroxypentanoic acid, consistent with the selective hydrogenation of levulinic acid under the mild experimental conditions. Reuse was also possible for the carbon based multifunctional catalyst (Ru/CNP500) with no activity loss during multiple recycles, similar to the bifunctional mixtures of (Ru/Csigma+Amberlyst 15) and (Ru/Csigma+NbP-bulk). However, the NbOPO₄ based bifunctional catalyst (Ru/NbP-bulk) demonstrated a steady activity loss (ca. 3-3.5%) with each recycle, most likely due to the sintering or loss of active Ru nanoparticles (FIG. 6(a)).

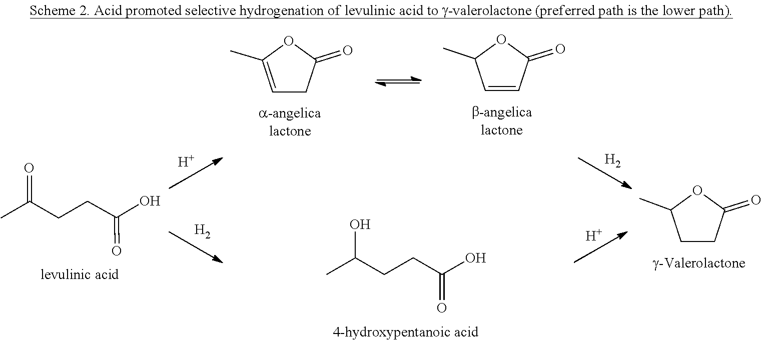

### Catalytic Activity in Continuous Flow

The low temperature hydrogenation activity of multifunctional Ru/CNP500 catalyst under continuous flow conditions was investigated. The stability of bi-functional mixtures of (Ru/Csigma+Amberlyst 15) and (Ru/Csigma+NbP-bulk) were also explored for the sake of comparison (Ru loading was 5 wt %, in all the catalysts). Catalytic experiments were conducted in a custom made down flow fixed-bed reactor allowing for a concurrent flow of H₂ and liquid feed (aqueous levulinic acid), keeping reaction conditions similar to those of batch operations. Representative results from fixed-bed experiments are summarized in Table 4, which confirm the exceptional catalytic performance of multifunctional Ru/CNP500 catalyst upon γ-valerolactone production, which was en par to the bifunctional mixtures of commercial Ru/Csigma with the acid co-catalysts, in terms of conversion and selectivity. The data presented in Table 4, also confirm the possibility of tuning levulinic acid conversion and γ-valerolactone yield/selectivity by simple adjustment of flow rate of liquid feed (liquid/weight hourly space velocity), gaseous H₂ (H₂/levulinic acid mole ratio) and reaction temperature and pressure. In terms of γ-valerolactone productivity

$$( {g}_{GVL} {g}_{Ru}^{- 1} {h}^{- 1} )$$

even though the blended catalytic systems outperformed the multifunctional Ru/CNP500 (Table 4), the later was more advantageous in terms of long term stability resulting from the enhanced stabilization of Ru-nanoparticles over a highly nitrogen doped carbon surface (see below) [19-21].

To evaluate the lifetime/stability of catalytic systems, catalytic reactions under fixed-bed conditions were continuously monitored at fixed liquid hourly space velocity, H₂/levulinic acid mole ratio and fixed temperature for at least 12 h or, until steady deactivation was observed (decay of γ-valerolactone selectivity or conversion). If no deactivation was observed during ~120 h operation, reaction conditions such as temperature, liquid hourly space velocity, H₂/levulinic acid mole ratio and liquid feed concentration were adjusted to evaluate the effect of these parameters on the stability of catalyst (see below). It is also important to emphasize here that our process/catalysts worked at H₂/levulinic acid (mole) ratio 4-17, much lower than a benchmark bifunctional catalyst (Ru@DOWEX) reported for continuous γ-valerolactone production, operating a with H₂/levulinic acid (mole) ratio 54-95 under comparable conditions. Comparison of the time on stream data obtained for hydrogenation of 0.48 M levulinic acid at 85° C. for the blended catalytic systems (Ru/Csigma+Amberlyst 15 and Ru/Csigma+NbP-bulk) with Ru/CNP500, further confirm the superiority of the multifunctional catalyst showing no signs of deactivation during 120 h operation, in contrast the bifunctional mixtures showed significant activity loss at ca. 10 h and ~35 h for (Ru/Csigma+NbP-bulk) and (Ru/Csigma+Amberlyst 15), respectively (FIG. 6(b)). Due to the low pH of feed, in this work we opted for deactivation studies at full conversion to eliminate the leaching of Ru species through the formation of Ru-carboxylate complexes with levulinic acid (pKa ~4.59) in liquid phase.

Nevertheless, the activity loss of bifunctional mixtures could be attributed mainly to the sintering of Ru nanoparticles, and also partially to the formation of metal sulfides under reducing H₂ atmosphere for the Ru/Csigma+Amberlyst 15 system, as ICP-OES analysis revealed no leaching of Ru species.

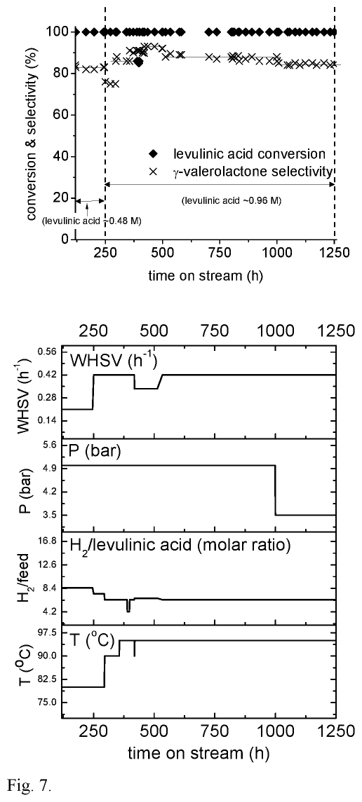

For Ru/CNP500 catalyst, extended stability tests were conducted with the same catalyst without any regeneration or pretreatment by varying reaction conditions to further investigate the influence of reaction parameters and feed concentration on catalyst stability. From the data presented in Table 4, and the time on stream plots for 5% Ru/CNP500 in FIG. 7, it could be seen that during 1250 h (52 days) operation, the levulinic acid conversion was stable and close ~100%, irrespective of the reaction temperature, pressure, weight hourly space velocity, and liquid feed concentration. Nevertheless, a slight decrease (ca. 10%) in γ-valerolactone selectivity was observed upon switching from 0.48 M to 0.96 M levulinic acid feed, selectivity could be increased to the desired levels ≥90% by either increasing the reaction temperature or reducing weight hourly space velocity of liquid feed. The decreased selectivity could be attributed to lack of sufficient moderate acid (phosphate) sites for cyclization of 4-hydroxypentanoic acid. Nevertheless, the ~100% levulinic acid conversion, observed was in good agreement with the kinetically favourable hydrogenation of carbonyl carbon of levulinic acid, and the high activity of small Ru-nanoparticles (~3.6 nm) deposited on the multifunctional carbon support CNP500 (Table 2 and 3). However, upon lowering the H₂/levulinic acid (mole) ratio to 4, conversion of levulinic acid reduced to 85%, γ-valerolactone selectivity was close to the desired levels (≥90%), most importantly both conversion and selectivity remained unaffected during ~30 h operation under these conditions FIG. 7. Reaction pressure was found have positive influence on the selectivity of the desired product (γ-valerolactone) as shown in Table 3 and FIG. 7 respectively, however use of H₂ pressures greater than 5 bar was avoided in this study. Overall, the results of extended stability test confirm the exceptional stability of multifunctional Ru/CNP500 (Ru loading was 5%) catalyst for continuous hydrogenation of aqueous levulinic acid (0.48 M to 0.96 M) to γ-valerolactone under mild process conditions.

Without wishing to be bound by any specific scientific theory the inventors believe that the exceptional catalytic performance and durability of the Ru/CNP500 catalyst can be attributed to enhanced stabilization of Ru-nanoparticles over the nitrogen surface defects, which prevent nanoparticle aggregation and leaching, thus inhibiting deactivation. Most importantly, unlike the bifunctional Ru catalysts incorporating unstable and reducible SO₃H groups as the acidic sites, the acidic phosphate sites of multifunctional Ru/CNP500 catalysts are non-reducible and chemically stable under the experimental conditions. In fact, it represents the most economical, and stable Ru-catalyst for energy efficient hydrogenation of levulinic acid. Furthermore, the unique ability of the catalyst to work with low pressure H₂ streams and low H₂/feed (mole) ratio may enable development of a small scale hydrogenation reactor for γ-valerolactone production, capable of directly utilizing renewable H₂ from water electrolysis.

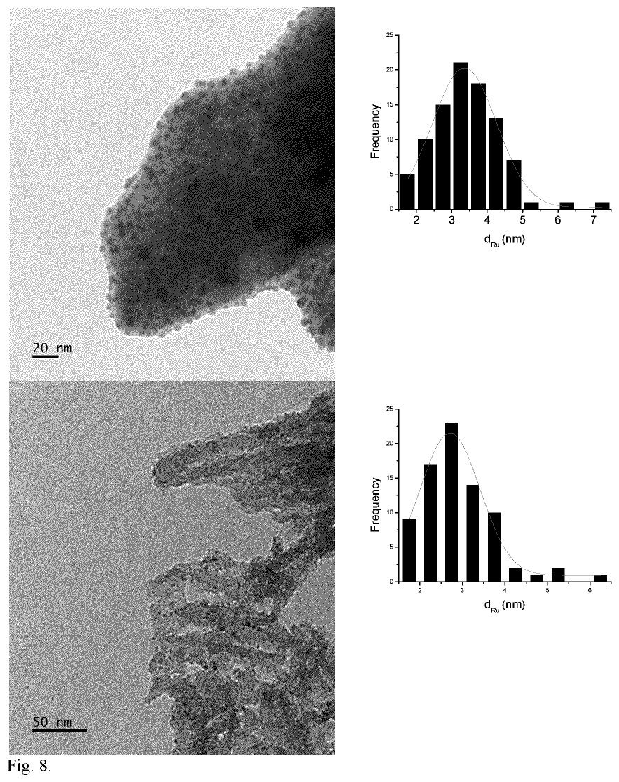

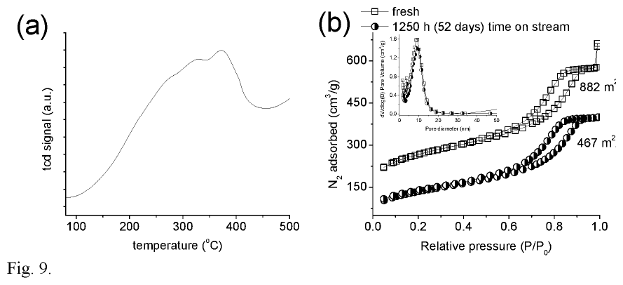

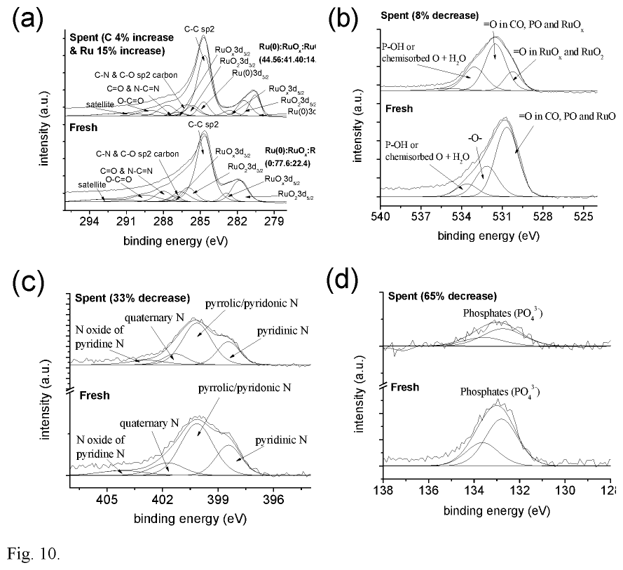

The experimentally observed activity, selectivity and stability of Ru/CNP500 catalyst was further supported from the characterization results of spent catalyst collected from the fixed bed after 1250 h time on stream, while the absence of Ru-species leaching was confirmed by ICP-OES analysis of collected liquid products at regular intervals. A comparison of the TEM images show that even after 52 days of continuous operation (~1250 h time on stream) under varying conditions of pressure, temperature, H₂ and feed flow rate, aggregation or sintering of nanoparticles was non-observable, (FIG. 8). However, NH₃-TPD patterns of the spent catalyst revealed a slight shift of the medium temperature desorption peak to a higher temperature, which could be attributed to the partial reduction of RuOₓ/RuO₂ species to metallic Ru (FIG. 9(a)). Deposition of carbonaceous impurities could also partially contribute to this shifting of desorption peak. Comparison of the textural properties obtained from N₂-physiorption confirm that the material retained its mesoporous structure, 53% of its specific surface area and 62% of its pore volume even after prolonged use (52 days) under varying experiential conditions. The slight reduction in porosity and specific surface area is most likely linked with the strongly chemisorbed oxygenated molecules on catalyst surface functional groups, which block access to the smaller micropores (Table 2 and FIG. 9(b)). Further confirmation on the hypothesis regarding the presence of adsorbed organic species and the exceptional stability of immobilized Ru-nanoparticles, were obtained from the XPS surface analysis. Comparison of the high resolution of XPS spectra Cls, Ols, N1s, P2p and Ru3d region of spent Ru/CNP500, with the as synthesized Ru/CNP500, also revealed only minor compositional changes upon the surface chemistry CNP500 even after 52 days upon continuous operation and γ-valerolactone production (FIG. 10). As expected, the Cls region revealed a slight increase in the intensity of C1s (C═O) peaks, reflected by a ca. 4% increase in the carbon content, consistent with the strong adsorption of γ-valerolactone molecules (FIG. 10(a)). A comparison of the Ols region, revealed a decrease in the intensity of Ols (═O) peak, which could be attributed to reduction of surface RuO₂/RuOₓ species to metallic Ru, the removal of surface oxygenates (such as COOH, OH) are unlikely considering the mild reaction conditions (FIG. 10(b)). The N1s region did not show a marked change and retained ca. 70% of its sites, even after 52 days continuous operation under reducing conditions (FIG. 10(c)). A comparison of P2p region revealed a significant reduction (ca. 65%) in the phosphate content, which could be attributed to hydrolytic cleavage of C—P or C—O—P linkages under hydrothermal environments (FIG. 10(d)).

Even so, the material retained adequate amount of Brønsted acidic phosphate sites to promote dehydration of 4-hydroxypentanoic acid to γ-valerolactone production, at ~1250 h time on stream. Most importantly, the acidic phosphate sites were non-reducible, even in the presence of excess H₂, at elevated temperatures (80-95° C.), which addresses the phenomena of permanent deactivation associated with the bifunctional Ru-catalysts based on sulfated supports. Finally, a comparison of the Ru3d peaks of spent and fresh Ru/CNP500 revealed no net decrease in the Ru content (FIG. 10(a)), in agreement with findings of ICP-OES analysis, while the presence of metallic Ru(0) in spent catalyst was attributed to the reduction of RuO₂/RuOₓspecies under reducing conditions. Based on the experimental findings, a plausible mechanism can be proposed for Ru/CNP500 catalyzed hydrogenation of levulinic acid to γ-valerolactone, where upon the Brønsted acid phosphate and Lewis acidic RuO₂/RuOₓ species facilitated the intramolecular esterification of the intermediate hydrogenated product 4-Hydroxypentanoic acid into γ-valerolactone under mild conditions (Scheme 3).

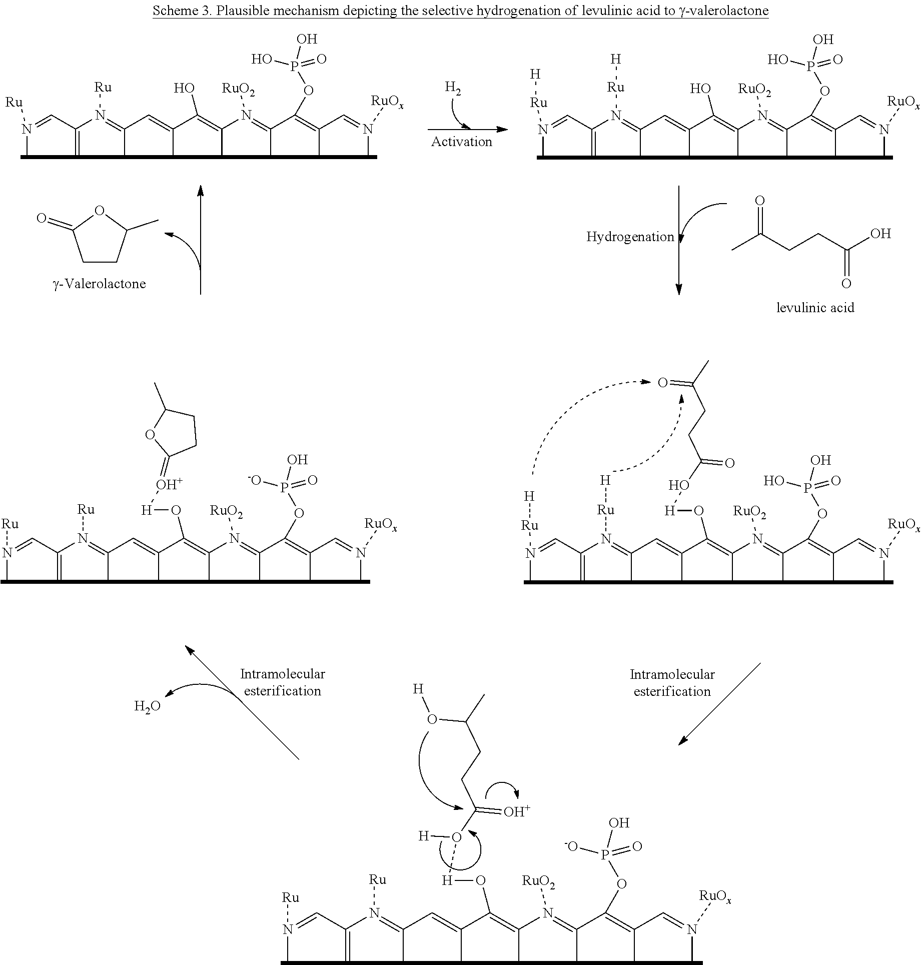

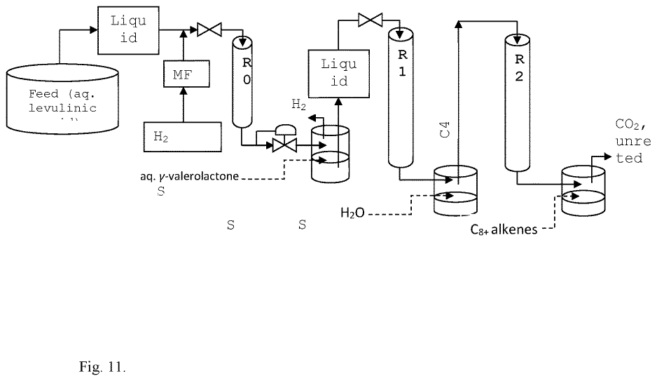

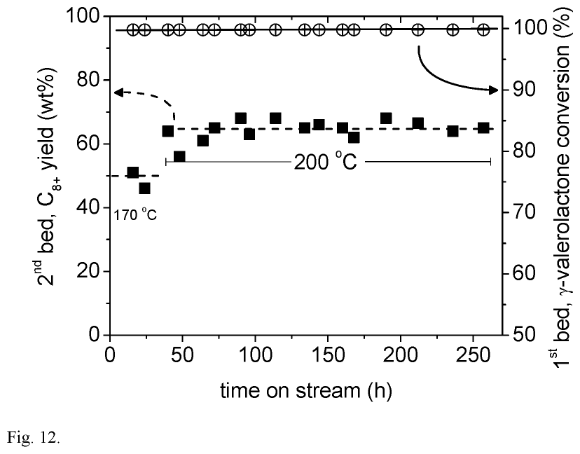

Furthermore, to confirm the commercial/industrial utility of the demonstrated process, e.g. upon the synthesis of synthetic hydrocarbons, we also examined the tandem conversion of the 9 wt % aqueous γ-valerolactone (or γ-valerolactone and 4-hydroxypentanoic acid) obtained over multifunctional Ru/CNP500 catalyst into C₈₊ branched olefins in a cascade process following a procedure similar to U.S. Pat. No. 8,410,326 B2 (Dumesic et al) and U.S. Pat. No. 8,975,461 B2 (Peters et al.) (FIG. 11). However, unlike the previous processes operating at high pressures (e.g. 36 bar) and concentrated feeds (30% or higher), our process operated at atmospheric pressure with a single pass, dual-bed reactor system comprising of commercial Silica-alumina (1^{st} bed) and Solid phosphoric acid (2^{nd} bed) (FIG. 12), also the process directly used the 9 wt % aqueous Q-valerolactone obtained from the hydrogenation reactor without any pretreatments. Interestingly, the use of lower pressure and a dilute γ-valerolactone feed prevented the deactivation of Silica-Alumina catalyst as observed in U.S. Pat. No. 8,410,326 B2 (Dumesic et al) (FIG. 11).

### CONCLUSIONS

In conclusion, a bio-waste (chitin) derived multifunctional Ru-catalyst (Ru/CNP500) incorporating moderately Brønsted acidic phosphate sites, Lewis basic N-sites and surface oxygenates is shown to exhibit exceptional activity, and stability upon selective hydrogenation of aqueous levulinic acid to γ-valerolactone under mild reaction conditions (80-95° C., stoichiometric or low H₂/feed ratio). The multifunctional catalyst outperformed the bifunctional Ru-catalysts based on NbOPO₄ and blended catalytic systems comprising of commercial Ru/carbon as the hydrogenation catalyst and NbOPO₄/Amberlyst 15 as a co-catalyst/acid promoter. Most importantly, the catalytic material demonstrated remarkable stability in a fixed-bed reactor under continuous flow conditions, maintaining high activity, and γ-valerolactone selectivity during ~1250 h time on stream operating at 3-5 bar, 80-95° C. and low H₂/levulinic acid ratio (4-17). The exceptional catalytic performance and stability of the multifunctional Ru-catalyst was attributed to the unique surface chemistry of carbon support that stabilized the Ru nanoparticles on surface nitrogen and oxygen defects, and promoted 4-hydroxypentanoic acid cyclization over acidic phosphate sites. Moreover, the acidic phosphate sites of the multifunctional Ru/CNP500 catalyst were non-reducible and chemically stable under the experiential conditions, unlike the previously reported bifunctional Ru-catalysts incorporating unstable and reducible SO₃H groups as the acidic sites. In fact, it represents the most economical, and stable Ru-catalyst for energy efficient hydrogenation of levulinic acid. Furthermore, the unique ability of the catalyst to work with low pressure H₂ streams and low H₂/feed (mole) ratio may enable development of a small scale hydrogenation reactor for γ-valerolactone production, capable of directly utilizing renewable H₂ from water electrolysis.

TABLE 1
Summary of textural and surface properties of carbon supports

| | ^{e} NH₃-acidity | | ^{f} CO₂- | basicity/ | specific | pore | pore | | |
|---|---|---|---|---|---|---|---|---|---|
| | (mmol/g) | | basicity | acidity | surface | volume | size | | ^{g} P |
| code | total | strong | (μmol/g) | (ratio) | area (m²/g) | (cm³/g) | (nm) | Hₒ | (wt. %) |
| ^{a}NbP-bulk | 1.159 | 0.663 | 0 | 0 | 132 | 0.14 | 4.09 | ≤−8.2 | 13.1 |
| ^{b}CPcomm | 2.44 | 0.65 | 0.00 | 0 | 1336 | 1.38 | 3.96 | ≤−3 | 0.12 |
| ^{c}CN650 | 1.19 | 0.17 | 0.044 | 0.037 | 342 | 0.34 | 4.24 | *3.75−4.74 | 0 |
| ^{d}CNP500 | 1.91 | 0.90 | 0.05 | 0.026 | 981 | 1.04 | 5.24 | ≤−3 | 0.18 |

^{a}bifunctional strongly acidic Nb₂O₅ & NbOPO₄ sites, characterization data compiled from ref. [25]
^{b}bifunctional moderately acidic —COOH, —OH & strongly acidic —OPO₄ sites
^{c}multifunctional acidic (—COOH & —OH) and basic (pyridinic, pyrrolic & N-oxide of pyridine nitrogen) sites
^{d}multifunctional acidic (—COOH, —OH & —OPO₄) and basic (pyridinic, pyrrolic , quaternary & N-oxide of pyridine nitrogen) sites
^{e} based on NH₃-TPD (total 70-450° C., strong 320-450° C.)
^{f} based on CO₂-TPD (30-150° C.)
*represents pKa values for carboxylic acid
^{g} based on EDX

TABLE 2
Summary of metal loading, NH₃-acidity, textural and metal particle
properties of supported Ru-catalysts utilized in this work

| | Ru | ^{a}NH₃- | Specific | Pore | Pore | | |
|---|---|---|---|---|---|---|---|
| | loading | acidity | surface | volume | size | ^{b}D | ^{b}d_{p, H2} |
| Catalyst | (wt %) | (mmol/g) | area (m²/g) | (cm³/g) | (nm) | (%) | (nm) |
| Ru/NbP-bulk | 2.5 | 1.159 | n.d | n.d | n.d | 75.7 | 1.76 |
| Ru/CPcomm | 5 | n.d | 1280 | 1.12 | 3.98 | 34.1 | 3.91 |
| Ru/CN650 | 5 | 3.32 | 318 | 0.319 | 4.28 | 46.8 | 2.85 |
| Ru/CNP500 | 1 | 2.1 | 942 | 0.95 | 5.01 | 50.2 | 2.25 |
| | 2.5 | 3.14 | 897 | 0.91 | 5.03 | 36.8 | 3.63 |
| | 5 | 4.27(4.17) | 882(467) | 1.02(0.61) | 5.8(5.29) | 23.1 | 5.70 |
| Ru/CNP500 IW | 5 | 1.23 | n.d | n.d | n.d | n.d | n.d |
| Ru/Csigma | 5 | 2.35 | 754 | 0.74 | 5.7 | 53.9 | 2.47 |

^{a}based on NH₃-TPD (total 70-450° C.)
^{b}based on H₂ pulse chemisorption, where D = dispersion and d_{p, H2} = average metal particle size
*values in parentheses ( ) represent NH₃-acidity, specific surface area, pore volume and pore size for catalyst after 1250 h time on stream

TABLE 3
Summary of the activity of different Ru catalysts for the catalytic
conversion of levulinic acid to γ-valerolactone in batch mode

| | | Ru | 4 h | | | |
|---|---|---|---|---|---|---|
| | | loading | Conversion | Selectivity (%) | | Specific rate |
| Entry | Catalyst | (wt %) | (%) | 4-HPA | GVL | (mol/min · g_{Ru}) |
| 1 | Ru/NbP-bulk | 2.5 | 100 | 0 | 100 | 2.87 × 10⁻² |
| 2 | Ru/CPcomm | 5 | 100 | 2 | 98 | 1.18 × 10⁻² |
| 3 | Ru/CN650 | 5 | 32 | 15 | 75 | 3.83 × 10⁻³ |
| 4 | Ru/CNP500 | 5 | 100 | 2 | 98 | 2.39 × 10⁻² |
| 5 | Ru/CNP500_IW | 5 | 63 | 3.5 | 90 | 6.91 × 10⁻³ |
| 6 | Ru/Csigma | 5 | 100 | 10 | 90 | 1.18 × 10⁻² |
| 7 | Ru/Csigma + NbP-bulk (0.1 g) | 5 | 100 | trace | 99.5 | 2.40 × 10⁻² |
| 8 | Ru/Csigma + Amberlyst 15 (0.1 g) | 5 | 100 | 0 | 100 | 2.40 × 10⁻² |

Reaction conditions: 0.25 g levulinic acid, 5 ml deionised H₂O, 15-35 mg catalyst (@0.34 mol % Ru), 5.5 ± 1 bar initial H₂, 80° C. (*25° C.), 400 rpm (stirring rate)

TABLE 4
Summary of representative experiments showing the catalytic activity of multifunctional Ru/CNP500 catalyst in comparison
to the bifunctional Ru/C and acid-co catalyst mixtures for the catalytic conversion of levulinic acid to γ-valerolactone under
continuous flow conditions.

| | | | | feed | | | | | | |
|---|---|---|---|---|---|---|---|---|---|---|
| | | P | T | conc. | ^{b}H₂/LA | ^{d}WHSV | ^{d}conversion | ^{e}selectivity (%) | | ^{f}productivity |
| Entry | ªCatalyst | (bar) | (° C.) | (M) | (ratio) | (h⁻¹) | (%) | GVL | 4-HPA | $( {g}_{G V L} {g}_{R u}^{- 1} {h}^{- 1} )$ |
| 1 | Ru/CNP500 | 5 | 85 | 0.48 | 17 | 0.105 | 100 | 96 | 4 | 2.1 |
| 2 | | 5 | 85 | 0.48 | 11 | 0.168 | 100 | 96 | 4 | 3.22 |
| 3 | | 5 | 85 | 0.48 | 9 | 0.21 | 100 | 96 | 4 | 4.03 |
| 4 | | 5 | 80 | 0.48 | 9 | 0.21 | 100 | 82 | 18 | 3.44 |
| *5 | | 5 | 80 | 0.96 | 7.5 | 0.42 | 100 | 75 | 25 | 6.38 |
| *6 | | 5 | 90 | 0.96 | 6.3 | 0.42 | 100 | 86 | 14 | 7.22 |
| *7 | | 5 | 95 | 0.96 | 6.3 | 0.42 | 100 | 89 | 11 | 7.56 |
| *8 | | 5 | 95 | 0.96 | 4 | 0.42 | 85 | 91 | 9 | 6.57 |
| *9 | | 3.5 | 95 | 0.96 | 6 | 0.42 | 100 | 84 | 16 | 7.04 |
| 10 | Ru/Csigma + Amberlyst | 5 | 85 | 0.48 | 8 | 0.26 | 98 | 100 | 0 | 20.52 |
| 11 | 15 (co-catalyst) | 5 | 85 | 0.48 | 16 | 0.13 | 100 | 100 | 0 | 11.11 |
| 13 | Ru/Csigma +NbP-bulk | 5 | 85 | 0.48 | 12 | 0.26 | 98 | 97 | 0 | 21.80 |
| 14 | (co-catalyst) | 5 | 90 | 0.48 | 12 | 0.13 | 100 | 100 | 0 | 11.14 |
| 15 | | 5 | 85 | 0.48 | 8.4 | 0.08 | 100 | 100 | 0 | 6.68 |

^{a}Ru loading was fixed at 5 wt % for all catalysts, and used without pre-reduction under H₂
^{b}molar ratio of H₂ to levulinic acid
${}^{c} WHSV ( {h}^{- 1} ) = \frac{levulinic acid flow rate ( {gh}^{- 1} )}{mass of catalyst ( g )}$ ^{d}levulinic acid HPLC conversion,
^{e}GC-FID selectivity of γ-valerolactone (GVL) and 4-hydroxypentanoic acid (4-HPA)
${}^{f} productivity = \frac{γ - valerolactone produced ( {gh}^{- 1} )}{mass of Ru in catalyst ( g )}$

1. A catalytic material based on a material comprising carbon, the catalytic material is loaded with Ru, wherein a surface of the catalytic material comprises a) Brønsted acidic phosphate groups, b) Lewis basic N-groups selected from the group consisting of Lewis basic pyridinic, pyrrolic and quaternary N-groups, and c) oxygen functional groups.

2. The catalytic material according to claim 1, wherein the content of Ru is 0.1-10 wt %.

3. The catalytic material according to claim 1, wherein the content of Ru is 1-6 wt %.

4. The catalytic material according to claim 1, wherein the Ru is present in the form of nanoparticles comprising Ru.

5. The catalytic material according to claim 1, wherein the NH₃-acidity is more than 1.5 mmol/g, measured by NH₃-TPD (temperature programmed desorption) (total 70-450° C.).

6. The catalytic material according to claim 1, wherein the CO₂-basicity is more than 0.03 μmol/g, measured by CO₂-TPD (temperature programmed desorption) (total 30-150° C.).

7. The catalytic material according to claim 1, wherein the specific surface area is at least 300 m²/g.

8. The catalytic material according to claim 1, wherein the pore volume is at least 0.3 cm³/g.

9. The catalytic material according to claim 1, wherein the catalytic material is based on a carbonized organic material.

10. The catalytic material according to claim 1, wherein the catalytic material is based on carbonized chitin.

11. The catalytic material according to claim 1, wherein the Brønsted acidic phosphate groups comprises NbOPO₄.

12. A system comprising a catalyst bed comprising the catalytic material according to claim 1, a heater and thermocouple, a pump for feeding levulinic acid to the catalytic bed, a pressure regulator for regulating the pressure in the catalytic bed/system, a valve for regulating inflow of H₂.

13. The system according to claim 12, wherein the system comprises a valve for regulating inflow of N₂.

14. The system according to claim 1, wherein the system comprises a gas-liquid separator for separating obtained liquid product from unreacted H₂.

15. A method for converting levulinic acid to γ-valerolactone comprising use of the catalytic material according to claim 1, the method comprising the steps of contacting the catalytic material with an aqueous solution of levulinic acid and hydrogen gas (H₂).

16. The method according to claim 15, wherein the H₂/levulinic acid mole ratio is in the interval 3-20.

17. The method according to claim 15, wherein the pressure during reaction is in the interval 1.5-10 bar.

18. The method according to claim 15, wherein the temperature is above 70° C. but below the boiling point of the aqueous levulinic acid solution at the pressure at which the reaction is carried out.

19. The method according to claim 15, wherein the catalytic material is contacted with the aqueous solution of levulinic acid and hydrogen gas batch-wise in a reaction vessel.

20. The method according to claim 15, wherein the catalytic material is contacted in a fixed-bed reactor under continuous flow conditions of aqueous solution of levulinic acid and hydrogen gas.

21. The method according to claim 15, wherein the levulinic acid is made from a raw material comprising cellulose.

22. The method according to claim 15, comprising further subsequent steps wherein the γ-valerolactone or γ-valerolactone/4-hydroxypentanoic acid mixtures are further converted into C₈₊ olefins in a cascade process at atmospheric pressure with a single pass, dual-bed reactor system comprising commercial silica-alumina (1^{st} bed) and solid phosphoric acid (2^{nd} bed).

23. γ-valerolactone or γ-valerolactone/4-hydroxypentanoic acid mixtures manufactured using the method according to claim 15.

24. A C₈₊ olefin manufactured with the method according to claim 22.

25. A method for preparing a catalytic material according to claim 1, the method comprising the steps of contacting an organic polymer comprising amine groups with phosphoric acid followed by a subsequent step of carbonizing the material under elevated temperature under self-generated atmosphere to provide a carbon based material wherein a surface of the material comprises a) Brønsted acidic phosphate groups, b) Lewis basic N-groups selected from the group consisting of Lewis basic pyridinic, pyrrolic and quaternary N-groups, and c) oxygen functional groups, where after a final loading step is performed where the material is mixed with an aqueous solution of a ruthenium salt where after the ruthenium salt is reduced so that small particles comprising elemental ruthenium are loaded on the carbon based material.

26. The method according to claim 25, wherein the organic polymer comprising amine groups is chitin.

27. The method according to claim 25, wherein the material is carbonized under elevated temperature of at least 500° C. in an atmosphere comprising an elevated concentration of N₂ compared to the normal atmosphere.
